(19) United States
(12) Patent Application Publication
(10) Pub. No.: US 2005/0249717 A1
(43) Pub. Date: Nov. 10, 2005

(54) Use of hyaluronidase for preventing and treating cardio vascular diseases

(76) Inventors: Gunther Burgard, Homburg (DE); Heinz Dieter Cullmann, Zweibrucken (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/296,418

(22) PCT Filed: May 25, 2001
(86) PCT No.: PCT/EP01/04940

§ 371 Date: Apr. 7, 2003

(30) Foreign Application Priority Data
May 29, 2000 (DE) 100 26 666.5

(51) Int. Cl. A61K 38/47

(52) U.S. Cl. 424/94.61

(57) ABSTRACT

The invention relates to use of the enzyme hyaluronidase in a dosage of at least 6,000 units per day for prevention and/or treatment of cardiovascular diseases whereby the hyaluronidase is administered intra venam over a period of at least four weeks. Hyaluronidase is particularly suited to treating coronary heart diseases as well as for preventive purposes in patients at risk of heart infarctions.

The present invention relates to the use of hyaluronidase for prevention and treatment of cardiovascular diseases such as coronary heart disease as well as other arterioscleroses as well as for prevention of restenosis in patients who have already been subjected to a coronary intervention. Coronary interventions include, for example, stent implantation, balloon dilatation, rotablation and bypass operations (arterio-occlusive disease treatments).

The present invention furthermore relates to prevention and treatment of acute cardiac infarction.

Cardiovascular diseases and the disturbance of well-being and the appearance of physical impairments as its sequelae have in recent years affected an increasing number of people. By now numerous medications have been developed in order to treat heart diseases. These cardiopharmaceuticals include complex compounds like vegetable extracts as well as individual chemical compounds such as those listed in the “2000 Red List”. But none of the currently marketed medications has shown really satisfactory results in treating cardiovascular diseases.

The application NL8102880 describes the use of 20,000-80,000 IU of hyaluronidase for treatment of arteriosclerosis, enhanced blood supply and embolism. The hyaluronidase is in these cases applied intra-arterially for some 15-30 minutes at intervals of three weeks.

The object of the present invention is to solve the technical problem of indicating an additional possibility of preventing and treating cardiovascular diseases in humans or animals.

This problem is solved by the present invention according to patent claim 1 by using hyaluronidase in a dosage of at least 6,000 units per day for prevention and/or treatment of cardiovascular diseases where the hyaluronidase is administered intra venam over a minimum period of four weeks, preferably in a bolus such as within ten seconds.

Additional advantageous and preferred embodiments of the invention are the subject of subclaims.

According to one embodiment of the invention, the hyaluronidase is used in a dosage of at least 12,000 units per day,

According to a further embodiment of the invention the hyaluronidase is used in a dosage of at least 15,000 units per day. However, it is also possible to work with a dosage of more than 100.000 units per day.

According to still another embodiment of the invention the hyaluronidase is additionally used in combination with camitin and/or CSE inhibitors and/or nootropics and/or blood supply enhancement agents and/or vitamins and/or cations.

It is obvious that combined use with simultaneous administration such as in the form of a combination medicine or by means of directly successive or temporally staggered administration of the various components can also be made. As a matter of course the combination medicine as well as the various components can be used in connection with pharmaceutically acceptable carriers or dilution agents.

According to yet another embodiment of the invention dosage can be spread out over at least two months and particularly preferable is over at least three months. However, this should only be understood as an example and the concrete dosage duration can naturally be varied in each individual case. Optimum results are obtained by administering a working dose of 12,000 units per day, five to seven times a week over a period of six to eight weeks.

As an example, the cardiovascular disease or disorder may involve a coronary heart disease (CHD), all forms of arteriosclerosis, carotid stenosis, stenosis of the arteries leading to the brain cardiac infarction, apoplexy, cardiac hypertrophy or fatty disease of the heart.

Additionally, the use of the enzyme for prevention of restenosis in patients who have already been subjected to a coronary intervention is indicated. Coronary interventions include, for example, stent implantation, balloon dilatation, rotablation and bypass operations (arterio-occlusive disease treatments).

It has moreover been found that according to the invention prevention or treatment of acute cardiac infarction can be carried out.

Here below the invention is described in more detail without limitation.

Hyaluronidase is in itself well-known in the state-of-the-art and belongs to the so-called β(1-4)-glycosidases. These enzymes are also designated as hyaluronate glycan hydrolases, EC 3.2.1.35 through 3.2.1.36. Hyaluronidase hydrolyses hyaluronic acid, a linear heteroglycan with alternating glucuronic acid and N-acetyl-glucosamine residues (acidic glycosaminoglycan (mucopolysaccharide)) and hyaluronate (the ionic form of hyaluronic acid) but also does the same to chondroitin sulphate.

The hyaluronidase can be derived from any source whatsoever and, for instance may be recovered from bovine protein (bovine type), leeches or bacteria (e.g. in the form of hyaluronate lyase). The hyaluronidase can also be of vegetable origin. The hyaluronidase can be isolated, for instance, from potatoes, tobaccos and peas. Genetic engineering techniques in the art can likewise be used to produce hyaluronidase.

Particularly preferred is any hyaluronidase which splits and thus depolymerises hyaluronic acid, chondroitin-4-sulphate, chondroitin-6-sulphate and mucotin sulphate where the most preferred hyaluronidase is an enzyme available commercially such as, by way of example, the hyaluronidase marketed under the trade name of Hylase® “Dessau” by the firm of Pharma Dessau.

The hyaluronidase can of course not only be used to therapy an already existing disorder or disease of the cardiovascular system but also for preventive treatment, that is prophylactically, of the indications cited above in order to avoid or delay the occurrence of such disorders.

For treatment and/or prevention of the disorders indicated above, a mixture of hyaluronidases of different origins can also be used.

Particularly well suited is any hyaluronidase for prevention in the case of patients at risk of cardiac infarction and/or for treatment of coronary heart disease. Here the enzyme can also be administered directly intra-coronarily in addition to intravenous administration.

Coronary heart disease is also known under the terms of “stenotic coronary sclerosis”, “degenerative coronary disease” and “ischaemic heart disease”. A pathophysiologic basis is reduction in blood supply caused by sclerosis or occlusion of coronary vessels and thus the supply of energy-providing substrate and oxygen to the heart muscle entailing a discrepancy between supply and demand and clearly noticeable under strain (limited coronary reserves). As syndromes the following are known; angina pectoris, cardiac infarction as well as left ventricular failure. A CHD can also progress for a long time asymptomatically while in the exercise ECG under certain conditions signs of a lack of blood supply can be shown.

The success of treatment can be checked, for example, by means of an ECG, an exercise ECG, a long-term ECG, echocardiography, stress echo as well as with myocardial scintigraphy and coronary angiography as well as coronary angloscopy. It has generally been shown that by means of administration according to the present invention blood supply to the coronary vessels was improved, to some extent even attaining complete healing.

The following examples will illustrate the invention without being limiting.

Numerous patients with cardiovascular diseases were treated, some of whom in particular suffered from coronary heart disease. The patients were treated for at least four weeks, preferably for at least two months, and most preferably for at least three months, with a hyaluronidase-containing preparation, preferably with the medicament Hylase® “Dessau”. When administering 6,000 units per day of this hyaluronidase preparation, intravenously, by way of example, five times (up to seven times) per week, after several weeks a significant reduction in complaints was observed with the patients.

Some patients were also treated with dosages of Hylase® “Dessau” in the range of 12,000 units per day, five to seven times per week, and with dosages extending up to 15,000 units per day, five to seven times per week. Besides the preferably used intravenous application, other kinds of application such as intramuscular or subcutaneous application, also led to success but where the intravenous application was the most effective method of administration.

When using other hyaluronidases than Hylase® “Dessau” other dosages may be required which the person skilled in the art can easily determine according to the practical circumstances of the case.

According to another aspect of the invention, treatment of cardiovascular diseases with hyaluronidase is done by way of exemplification in combination with camitin. By using this combination, cardiac hypertrophy and fatty disease of the heart can be counteracted and thus the risk of an infarction can be significantly reduced.

Biochemically, the use according to the invention is directly connected with the degradation of fatty acids. Since the fatty acid synthesis preferably takes place in the cytoplasm while oxidative breakdown of fatty acids takes place in the mitochondria, the fatty acids to be broken down must first migrate from the cytoplasm to the mitochondria. Camitin (4-trimethylamino-3-hydroxybutyric acid) assists in such migration and can react both with acetyl CoA as well as with acyl CoA with the formation of acetyl-carnitin or acyl-carnitin. The formation of camitin-activated acetic acid or fatty acid is catalysed by means of acyl-CoA-camitin-transferase of which there are two distinct types (for acetyl transfer as well as for acyl transfer). By substituting the acetyl residue for a fatty acid residue a carnityl-fatty acid compound is produced which can pass through the mitochondria membrane. This explains the favourable effect of camitin on fatty acid oxidation. After entering the mitochondria, the fatty acid is transferred from the acylcarnitin to the CoA and the camitin is then again available for fatty acid transport.

Additional active ingredients neither possessing any coenzyme function nor demonstrable as essential components for humans but being indispensable cellular components and to some extent performing functions as yet not researched are collectively referred to as vitamin-like active agents. They to some extent influence the permeability of membranes and capillaries.

These include, besides carnitin:
Meso-inositol (myo-inositol)
Essential fatty acids (vitamin F)
Flavanoids (vitamin P)

A further aspect of the invention relates to the use of hyaluronidase in combination with so-called CSE inhibitors (CSE=cholesterol synthesising enzymes) such as pravasin and all other so-called “statins”. In this case it is unambiguously proven (e.g. in the WCS study) that pravasin in particular and other statins as well have a cardio-protective effect Additionally, they act against arteriosclerosis precisely as hyaluronidase does. The purpose of the combination is to achieve optimisation of therapy by means of the two substances' synergies.

Additional substances which may favourably influence the effect of hyaluronidase and which can be used in combinations according to the invention:
1) Magnesium and other cations
2) Nootropics (e.g. piracetam)
3) Agents enhancing blood supply (e.g. pentoxifillin, ginkgo, etc.)
4) Various vitamins (e.g. vitamin C)
5) Substances increasing NO synthesis
6) Glycoprotein antagonists such as eptifibatid
7) Plasminogen activators such as altelase, retelase, etc. In case of acute coronary thrombosis (cardiac infarction)

Use according to the invention is furthermore suitable for the following indications:
1) Cerebral sclerosis
2) Multiple sclerosis
3) Oncology: improvement of resorption of cytostatics in diseased tissue
4) Ophthalmology (glaucoma)
5) Diabetic mlcro-angiopathy
6) Increase in fertility in men and women

1-6. (canceled)

7. A method of preventing and/or treating cardiovascular disease, which method comprises intravenously administering to a patient at least 6,000 units per day of hyaluronidase for at least four weeks, whereupon cardiovascular disease in the patient is treated and/or prevented.

8. The method of claim 7, which method comprises intravenously administering to the patient at least 12,000 units per day of hyaluronidase.

9. The method of claim 8, which method comprises intravenously administering to the patient at least 15,000 units per day of hyaluronidase.

10. The method of claim 7, which method further comprises administering one or more agents selected from the group consisting of carnitin, a cholesterol synthesizing enzyme (CSE) inhibitor, a nootropic, a blood supply enhancer, a vitamin, and a cation.

11. The method of claim 8, which method further comprises administering one or more agents selected from the group consisting of carnitin, a CSE inhibitor, a nootropic, a blood supply enhancer, a vitamin, and a cation.

12. The method of claim 9, which method further comprises administering one or more agents selected from the group consisting of carnitin, a CSE inhibitor, a nootropic, a blood supply enhancer, a vitamin, and a cation.

13. The method of claim 7, which method comprises administering hyaluronidase for at least two months.

14. The method of claim 8, which method comprises administering hyaluronidase for at least two months.

15. The method of claim 9, which method comprises administering hyaluronidase for at least two months.

16. The method of claim 10, which method comprises administering hyaluronidase and one or more other agents for at least two months.

17. The method of claim 11, which method comprises administering hyaluronidase and one or more other agents for at least two months.

18. The method of claim 12, which method comprises administering hyaluornidase and one or more other agents for at least two months.

19. The method of claim 7, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

20. The method of claim 8, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

21. The method of claim 9, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

22. The method of claim 10, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

23. The method of claim 11, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

24. The method of claim 12, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

25. The method of claim 13, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

26. The method of claim 14, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

27. The method of claim 15, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

28. The method of claim 16, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

29. The method of claim 17, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

30. The method of claim 18, wherein the cardiovascular disease involves coronary heart disease, arteriosclerosis, carotid stenosis, stenosis of arteries leading to the brain, cardiac infarction, apoplexy, cardiac hypertrophy, or fatty disease of the heart.

31. A method of preventing and/or treating cardiovascular disease, which method comprises administering to a patient at least 6,000 units per day of hyaluronidase for at least four weeks whereupon cardiovascular disease in the patient is treated and/or prevented.
